(19) United States
(12) Patent Application Publication
(10) Pub. No.: US 2010/0222549 A1
(43) Pub. Date: Sep. 2, 2010

(54) DECHALCOGENATIVE METHODS FOR THE PREPARATION OF ALLYLIC SULFIDES

(76) Inventors: David Crich, Grosse Point Park, MI (US); Maheswaran Karatholuvhu, Detroit, MI (US); Venkataramanan Krishnamurthy, Tamil Nadu (IN); Thomas K. Hutton, Decatur, IL (US); Franck Brebion, Paris (FR); Venkataraman Subramanian, Tamil Nadu (IN)

Correspondence Address: Olson & Cepuritis, LTD., 20 NORTH WACKER DRIVE, 36TH FLOOR, CHICAGO, IL, 60606, US

(21) Appl. No.: 12/451,193

(22) PCT Filed: Apr. 29, 2008
(86) PCT No.: PCT/US08/05471

§ 371 Date: May 10, 2010

Related U.S. Application Data
(60) Provisional application No. 60/926,917, filed on Apr. 30, 2007.

(51) Int. Cl. C07K 7/06; C07C 319/14; C07H 15/14; C07K 5/037; C07K 5/083; C07C 319/24; C07B 45/06

(52) U.S. Cl. 530/328; 568/59; 568/58; 560/153; 568/38; 536/18.2; 530/332; 530/331; 568/26; 204/157.76

(57) ABSTRACT

A dechalcogenative method for the preparation of an allylic sulfide comprises contacting an activated chalcogenide of Formula (I) with a thiol of Formula (II) for a period of time sufficient to form an intermediate of Formula (III), and supplying sufficient activation energy to the intermediate of Formula (III), in a suitable solvent, preferably in the absence of a phosphine or other thiophile, to induce a [2,3]-sigmatropic rearrangement therein to form an allylic sulfide of Formula (IV), with concomitant loss of chalcogen Z, as set forth in the following reaction scheme, wherein X is an activating group selected from the group consisting of CN, S-pyridyl, S-heteroaryl, SO2-aryl, and SO3Y; Y is an alkali metal ion; Z is Se or S; R1, R2, R3, R4, and R5 are each independently H or a hydrocarbon moiety; and R is an organic moiety.

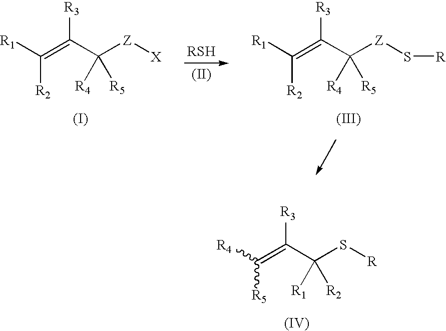

### STATEMENT OF GOVERNMENT INTEREST

A portion of the work described herein was supported by government-sponsored grants from the National Institutes of Health, Grant Nos. AI 56575 and GM 62160. The United States government has certain rights in this invention.

### FIELD OF THE INVENTION

The present invention relates to methods of preparing allylic sulfides. More particularly, the present invention relates to methods of preparing allylic sulfides utilizing a [2,3]-sigmatropic rearrangement of a selenosulfide or disulfide compound.

### BACKGROUND OF THE INVENTION

The development of methods for the functionalization of biopolymers, especially peptides and proteins, under the mildest possible conditions, dubbed ligation, is a current frontier in organic chemistry.¹⁻¹³ If such methods are to be truly useful and applicable to biochemical and biological systems, very high chemoselectivity, stability, and compatibility with protic solvents, and even aqueous media, are absolute requirements. The azide group¹⁴ has proven to be very versatile in this respect and forms the basis for two of the more important ligation methods, namely the Staudinger ligation¹⁵⁻²⁸ and Click chemistry,²⁹⁻⁴⁰ as well as of a variety of other reactions useful in this context.⁴¹⁻⁴⁷ Other methods include those relying on the chemoselective condensation of hydroxylamines and hydrazines with aldehydes and ketones, and related reactions, in aqueous solution.^{1, 2, 48-51}

Thiols, and in particular cysteine residues, have proven popular targets for chemoselective ligation.^{1, 2, 52} Native Chemical Ligation for the synthesis of peptides and proteins, and its enzyme-promoted biochemical equivalent, expressed protein ligation,⁵³⁻⁵⁵ are such reactions, which take advantage of the sulfhydryl (i.e., thiol) group and which use this group to great advantage in the highly chemoselective formation of amide bonds in aqueous solution.^{10, 26, 56-74} Another thiol-based method, the selective formation of mixed disulfides, is both one of the oldest and most enduring of ligation methods.^{1, 2, 75-81} The mildness of the disulfide ligation and its established chemoselectivity for the cysteine thiol in the presence of all the proteinogenic amino acids stands in stark contrast to the various other methods for cysteine functionalization, most of which involve the capture of the cysteine thiol by electrophilic species, and which consequently have obvious potential chemoselectivity issues.^{1, 2, 82} The practicality of the disulfide ligation, with its direct applicability to cysteine-containing peptides, also contrasts with the various ingenious indirect methods that have been developed for the preparation of S-functionalized cysteine derivatives,⁵² including, for example, the Michael addition of thiols to dehydroalanine units,⁸³ the alkylation of thiolates with peptide-based β-halo-alanine units,⁸⁴⁻⁸⁶ and other electrophiles,^{87, 88} the opening of peptide-based aziridines by thiolates,^{89, 90} and the synthesis of peptides with previously functionalized cysteine building blocks,⁹¹⁻⁹³ each of which requires the synthesis of modified peptides. The many advantages of the disulfide ligation are offset, however, by its impermanence, which results from the lability of the disulfide bond in the presence of thiols and other reducing agents.

Consideration of the practical advantages of the disulfide ligation, and the disadvantages of its impermanence, led us to investigate methods for rendering it permanent. In this regard, we have found that the allylic seleneosulfides and disulfides of complex molecules such as peptides and carbohydrates can undergo efficient dechalocogenative rearrangement to allylic sulfides under appropriate conditions.^{94, 95}

The phosphine-promoted desulfurative allylic rearrangement of diallyl disulfides to give allyl sulfides⁹⁶⁻¹⁰⁰ proceeds by way of a [2,3]-sigmatropic rearrangement via a diallyl thiosulfoxide intermediate^{101, 102} and, thus, is related to the well known Evans-Mislow rearrangement of allylic sulfoxides.¹⁰³ This dechalcogenative rearrangement, which may also be induced to operate in the reverse direction on treatment of allyl sulfides with elemental sulfur,¹⁰⁴ and which is potentially important in the chemistry of essential oils derived from garlic,¹⁰⁵ appeared to us to hold promise as a convenient means of providing a permanent ligation from a disulfide moiety, if it could be caused to function at ambient temperatures in protic solvents. The seminal work of Höfle and Baldwin revealed that the rearrangement is rapid in benzene at room temperature provided that the presumed thiosulfoxide intermediate is removed from the equilibrium. Thus, the diallyl disulfides 1 and 2 rearrange via the thiosulfoxides 3 and 4 to the thermodynamically more stable disulfides 5 and 6 via two sequential sigmatropic rearrangements with half-lives of 79 and 14 minutes, respectively, at 24° C. (Scheme 1)¹⁰¹

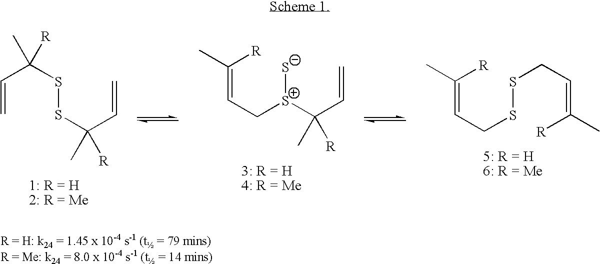

As shown in Scheme 2, in the absence of a thiophile, simple alkyl allyl sulfides 7 and 8 were found by Höfle and Baldwin to be stable at room temperature and could be purified by vacuum distillation, whereas 10 and 11 were reported to lose sulfur spontaneously at room temperature, albeit without mention of a timescale for this process.¹⁰¹ A comparable dependence of rearrangement rate on substituent pattern was observed by Moore and Trego in their early work on the reaction.⁹⁶ Pseudo first order rate constants were measured by Höfle and Baldwin for the reaction of the alkyl allyl disulfides 7-11 with triphenylphosphine in benzene at 60° C. leading to the conclusion that increased bulk around the thiosulfoxide reduces its concentration in the equilibrium and so retards reduction.¹⁰¹ While this remains possible, it is more likely that the equilibrium is shifted more toward the thiosulfoxide when a more highly substituted alkene is formed at the expense of a less substituted one, and when a more highly substituted C—S bond is replaced by a less substituted one.¹⁰¹

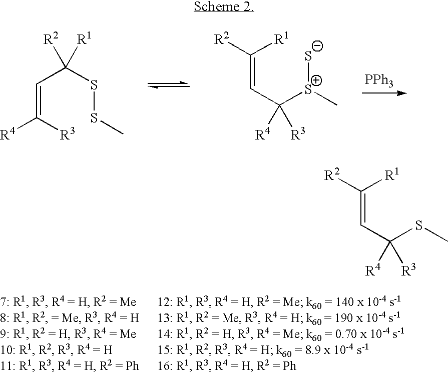

A single example of the corresponding deselenative rearrangement of a diallyl diselenide, that of di(geranyl) diselenide to geranyl linalyl selenide, was reported to proceed with a half life of approximately 2.5 hours at 25° C. on exposure to excess triphenylphosphine in chloroform, thereby indicating the selenium version of this reaction to be considerably faster than the original sulfur protocol.¹⁰⁶ Guillemin and coworkers subsequently were able to obtain crude preparations (˜80% pure) of diallyl diselenide, dicrotyl diselenide, and diprenyl diselenide, characterize them spectroscopically, and study their reactions with tributylstannane, indicating such diselenides to have at least moderate stability at room temperature.¹⁰⁷

### SUMMARY OF THE INVENTION

The present invention provides a dechalcogenative method for the preparation of an allylic sulfide, which is particularly well suited for use with complex molecules such as peptides and carbohydrates. The resulting allylic sulfides are useful as intermediates in a number of chemical transformations in the preparation of drugs and biochemical reagents. The method comprises contacting an activated chalcogenide of Formula (I) with a thiol of Formula (II) for a period of time sufficient to form an intermediate of Formula (III). Sufficient activation energy is then supplied to the intermediate of Formula (III) in a suitable solvent (e.g., an alcohol, an aqueous buffer or an aqueous buffer mixed with an organic solvent), preferably in the absence of a phosphine reagent, to induce a [2,3]-sigmatropic rearrangement, thereby forming an allylic sulfide of Formula (IV), with concomitant loss of chalcogen Z, as set forth in Reaction Scheme (A).

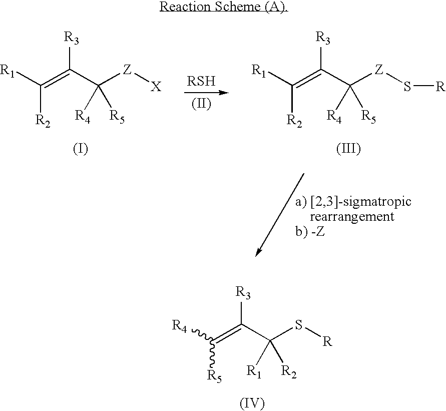

In the formulas set forth in Reaction Scheme (A), X is an activating group selected from the group consisting of CN, S-pyridyl, SO₂-aryl (preferably SO₂Ph), and SO₃Y; Y is an alkali metal (e.g., Na or K); Z is Se or S; R¹, R², R³, R⁴, and R⁵ are each independently H, a hydrocarbon moiety, or a substituted hydrocarbon moiety; and R is an organic moiety. In a preferred embodiment, X is an activating group selected from the group consisting of S-pyridyl, S-heteroaryl, SO₂-aryl, and SO₃Y; Y is an alkali metal ion; Z is S; R¹, R², R³, R⁴, and R⁵ are each independently H or a hydrocarbon moiety; and R is an organic moiety.

Hydrocarbon moieties can be saturated or unsaturated, and include, for example, alkyl groups, substituted alkyl groups, aryl groups, S-heteroaryl groups, and substituted-aryl groups, most preferably alkyl groups. Substituted hydrocarbon moieties can be alkyl and/or aryl groups substituted with one or more functional group, including a hydroxyl group, an ether group, an amino group, a carboxyl group, an ester group, an amide group, a carbonyl group, an acetal group, a hemiacetal group, a thiol, a thioether, a phosphonate group, a phosphate group, a phosphate ester group, a phosphoramide group, a halide, a heterocyclic group, a fully or partially fluorinated alkyl group, a polyethylene glycol group, a carbohydrate or derivatized carbohydrate, a peptide, a chromophoric group, a fluorophoric group, and the like, as desired.

Preferred organic moieties, derived from thiol-substituted compounds, include alkyl groups, substituted-alkyl groups, aryl groups, substituted-aryl groups, amino acids, carbohydrates, peptides, or groups consisting of two or more of the foregoing bound together. Particularly preferred organic moieties are complex molecules such as amino acids, peptides (e.g., polypeptides and proteins), carbohydrates (e.g., sugars and polysaccharides), nucleic acids, and peptide nucleic acids, more preferably peptides.

Preferably, the thiol of Formula (H) is a thio-substituted peptide or a thio-substituted carbohydrate.

Any suitable solvent can be used in the present invention. Preferred solvents include polar solvents such as acetonitrile, anhydrous or aqueous lower alcohols (e.g., C₁-C₃ alcohols such as methanol, ethanol, and isopropanol), an aqueous buffer, an aqueous buffer mixed with an organic solvent, and appropriate mixtures thereof.

The activation energy required to drive the rearrangement to completion is preferably provided by applying heat (e.g., in a refluxing solvent or under microwave irradiation), with or without a catalyst, such as an amine (e.g., piperidine) to facilitate the rearrangement.

### DETAILED DESCRIPTION OF PREFERRED EMBODIMENTS

The following discussion describes preferred aspects of the present invention, and is not to be construed as limiting the scope thereof.

As used herein and in the appended claims, the term “substituted” as applied to hydrocarbons and other organic molecules, means that the hydrocarbon or other organic molecule includes one or more functional group such as a hydroxyl group, an ether group, an amino group, a carboxyl group, an ester group, an amide group, a carbonyl group, an acetal group, a hemiacetal group, a thiol, a thioether, a phosphonate group, a phosphate group, a phosphate ester group, a phosphoramide group, a halide, a heterocyclic group, and the like, as desired.

As used herein and in the appended claims, the term “alkyl” encompasses saturated hydrocarbon groups, as well as non-aromatic unsaturated hydrocarbon groups, such as alkenyl groups and alkynyl groups.

As used herein and in the appended claims, the term “peptide” and grammatical variations thereof, refers to compounds including at least two amino acid residues bound together by a peptide (amide) bond, including dipeptides, oligopeptides, polypeptides, proteins, and any derivatives thereof (e.g., including a protecting group, a carbohydrate group, a lipid group, and the like bound to an amino acid residue in the peptide).

Preliminary Studies. As shown in Table 1, allylic pyridyl sulfides,^{1, 2, 108} allylic thiosulfonates,⁸¹ and allylic Bunte salts (S-allyl thiosulfates)^{109, 100} all proved suitable for the transfer of allylic sulfides to a range of simple thiols. The phosphine-promoted rearrangement of the resulting disulfide, however, was not so facile in all cases. For example, it was necessary to heat the alkyl geranyl disulfide 18 with triphenylphosphine in toluene at reflux in order to achieve efficient conversion to the rearranged linalyl sulfide 19 (Table 1, entry 1). It is important to note that, consistent with the goal of developing a practical process suitable for application to biologically relevant systems, the reactions were carried out with a minimal excess of phosphine, unlike the work of Höfle and Baldwin.¹⁰¹ The difference in the amount of phosphine presumably accounts for the differences in reaction times and temperatures observed. Under the same conditions the phenyl geranyl disulfide 20, however, gave the unrearranged phenyl geranyl sulfide 21 (Table 1, entry 2), thereby drawing attention to the relatively fine dividing line between the desired dechalcogenative allylic rearrangement and the better known nucleophilic removal of a sulfur atom from a disulfide, which is an undesirable side reaction.¹¹¹

As is evident from the comparison of entries 1 and 2 in Table 1, the replacement of an alkylthiyl moiety by an arylthiyl group is sufficient to tip the balance in favor of nucleophilic attack by the phosphine on the native disulfide. Entry 3 of Table 1 illustrates how the replacement of triphenylphosphine by the more nucleophilic hexaethylphosphoramide enabled the reaction temperature to be reduced to ambient room temperature, but with continued formation of the simple desulfurization product. Finally, Table 1, entry 4, indicates that the use of hexaethylphosphoramide is not preferred, at least in the context of amino acid and peptide-based systems, owing to both transesterification and racemization evidenced by incorporation of deuterium from the solvent at the α-center.

TABLE 1
Thiol + Donor → Disulfide
Disulfide + PR₃ → Product

| | | | Disulfide | | Temp | | Product |
|---|---|---|---|---|---|---|---|
| Entry | Thiol | Donor | (% yield) | Solv | (° C.) | PR₃ | (% yield) |
| 1 | CH₃(CH₂)₇SH | 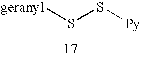 | 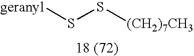 | A | 110 | D | 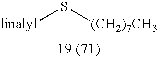 |
| 2 | PhSH | 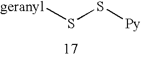 | 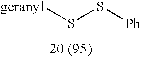 | A | 110 | D | 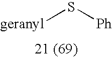 |
| 3 | PhSH | 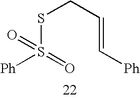 | 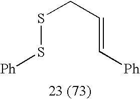 | B | RT | E | 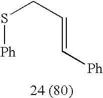 |
| 4 | 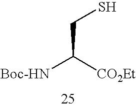 | 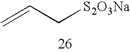 | 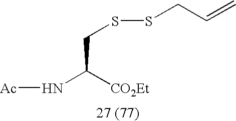 | C | RT | E | 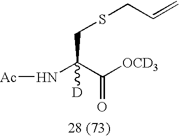 |

A = toluene;
B = hexadeuteriobenzene;
C = tetradeuteriomethanol;
D = triphenylphosphine;
E = hexaethylphosphoramide
RT = ambient room temperature

Allylic Selenosulfide Rearrangement. The clean formation of simple mixed alkyl selenosulfides can be realized at room temperature by the reaction of Se-alkyl seleno-Bunte salts with thiols.^{112, 113} Accordingly, a number of Se-allyl seleno Bunte salts were prepared by the reaction of potassium seleno sulfate, prepared in situ from potassium sulfite and selenium powder, with allyl halides. These compounds were typically orange crystalline solids that, while not indefinitely stable, could be handled in air at room temperature. Reaction with a range of aliphatic thiols then resulted in the formation of a series of selenosulfides, that could be readily detected by thin layer chromatography and/or NMR spectroscopy, but which were allowed to undergo the subsequent deselenative rearrangement, either with or without the addition of phosphine as required (Scheme 3, Table 2).

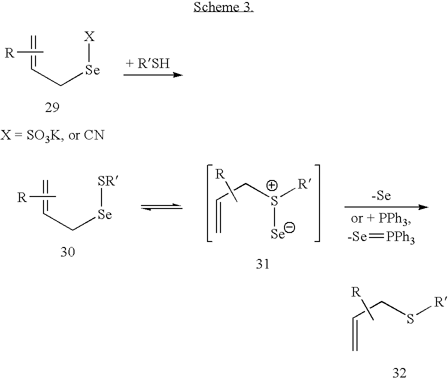

In a preferred embodiment of the present invention, the allyl selenosulfides are prepared by the reaction of allyl selenocyanates¹⁰⁷ with thiols, a process that also takes place readily at room temperature (Scheme 3), and which had been reported for the formation of simple diaryl selenosulfides.¹¹⁴ The formation of allyl selenosulfides from the reaction of allylselenols with disulfides was not investigated because of anticipated difficulties in the preparation and handling of the allylic selenols.¹⁰⁷ Overall, it is clear from Table 2 that allyl selenocyanates can generally be prepared in higher yields than the corresponding Se-allyl seleno Bunte salts, but that in most cases both selenenylation systems perform the transfer of the Se-allyl group to the thiol comparably well, as evidenced by the yield of the subsequent rearrangement products.

It is of some interest to note that the primary allylic selenocyanates prepared and employed in this study were readily handled at room temperature and showed no tendency to undergo rapid rearrangement. This observation parallels that of Riague and Guillemin, who had earlier prepared several of the same allylic selenocyanates and reported their purification by vacuum distillation,¹¹⁵ but it stands in contrast to the reported chemistry of simple allylic thiocyanates which are reported to undergo [2,3]-sigmatropic rearrangement to the corresponding allylic isothiocyanates in a matter of hours at room temperature.¹¹⁶ Carbohydrate-based allylic thiocyanates, however, require higher temperatures for rearrangement.¹¹⁷ Based on the work of Sharpless with 2-methyl-3-selenocyanato-1-heptene, secondary and presumably tertiary selenocyanates can be expected to undergo a [1,3]-sigmatropic rearrangement to their primary regioisomers.¹⁰⁶

TABLE 2

| | | | Disulfide | | | Product | |
|---|---|---|---|---|---|---|---|
| | | | % Yield | | | % Yield from | |
| | | | from Donor | | | Disulfide * | |
| | | | for X= | | | for X= | |
| Entry | Thiol | Donor | SO₃⁻K⁺ | CN | Product | SO₃⁻K⁺ | CN |
| 1 | CH₃(CH₂)₁₅SH | 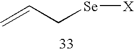 | 56 | 84 | 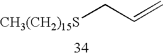 | 74 (0) | 70 (0) |
| 2 | 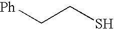 | 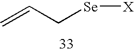 | 56 | 84 | 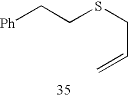 | 70 (0) | 70 (0) |
| 3 | 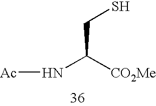 | 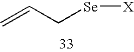 | 56 | 84 | 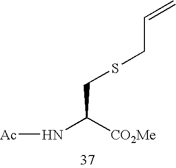 | 61 (2.5) | 80 (0) |
| 4 | 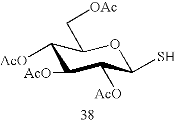 | 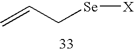 | 56 | 84 | 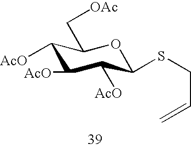 | 50 ★ (2.5) | 55 ♦ (2.5) |
| 5 | 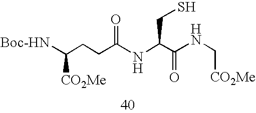 | 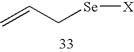 | 56 | 84 | 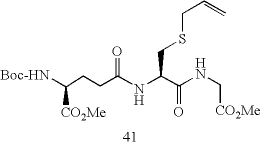 | 58 (2.5) | 75 (0) |
| 6 | 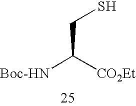 | 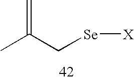 | 58 | 73 | 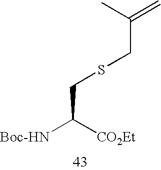 | 62 (2.5) | 68 (0) |
| 7 | 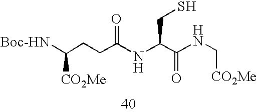 | 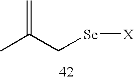 | 58 | 73 | 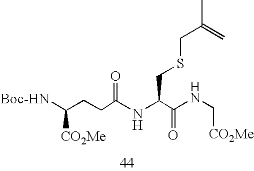 | 60 (2.5) | 71 (0) |
| 8 | 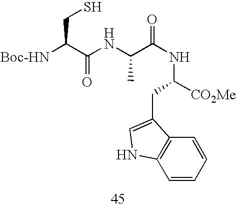 | 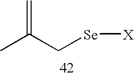 | 58 | 73 | 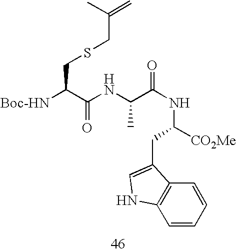 | 60 (2.5) | 65 (0) |
| 9 | CH₃(CH₂)₁₅SH | 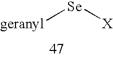 | 40 | 80 | 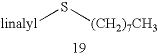 | 62 (2.5) | 70 (2.5) |
| 10 | 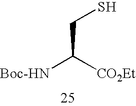 | 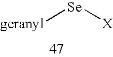 | 40 | 80 | 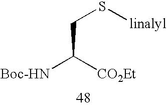 | 46 (2.5) | 66 (2.5) |
| 11 | 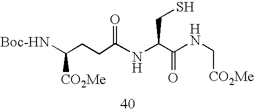 | 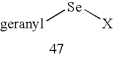 | 40 | 80 | 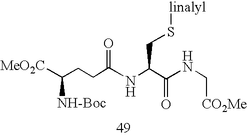 | 43 (2.5) | 55 (2.5) |
| 12 | 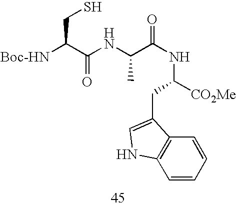 | 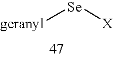 | 40 | 80 | 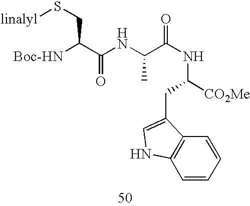 | 51 (2.5) | 50 (2.5) |
| 13 | 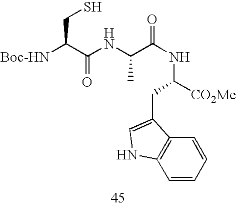 | 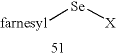 | 35 | 85 | 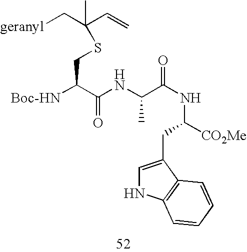 | 47 (2.5) | 40 (2.5) |
| 14 | 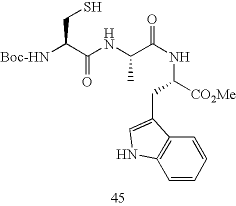 | 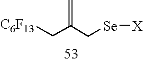 | 28 | 75 | 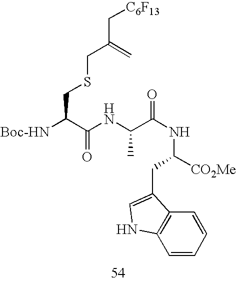 | 31 (2.5) | 65 (2.5) |

In Table 2, all reactions were conducted in MeOH at room temperature unless otherwise specified;.
* the number of equivalents of triphenylphosphine used in the rearrangement is shown in parenthesis ( ) below the product % yield;
♦ the rearrangement was run at 65° C.;
★ the rearrangement was run in CDCl₃ at 65° C.

The transfer of a simple allyl or methallyl group did not necessitate the addition of phosphine and proceeded spontaneously over a 2 hour period at room temperature. In the case of geranyl and farnesyl Se-Bunte salts and selenocyanates, proceeding with allylic rearrangement to the linalyl and nerolidyl sulfides, respectively, spontaneous rearrangement was not observed and it was necessary to add phosphine to provoke the loss of selenium. The more difficult rearrangement of the geranyl and farnesyl systems corresponds to the pattern observed originally with the allylic disulfides (Scheme 2), with the prenyl system 9 undergoing the phosphine-promoted rearrangement least rapidly. This is likely the consequence of the formation of the less stable isoprenyl substitution pattern. The anomeric thiol 38 rearranged slowly over a period of several days at room temperature in the presence of phosphine, and much more rapidly at 65° C. The fluorous substituted methallyl system 53, unlike the simple methallyl transfer reactions, necessitated the addition of phosphine in order to proceed at room temperature. These latter two observations are readily understood in terms of the mechanism of Scheme 3, with the formation of the intermediate selenosulfoxide disfavored by either an electron-withdrawing substituent on the allyl group or by the involvement of the sulfur atom in an exo-anomeric type interaction that serves to remove electron density from sulfur. When the reaction takes place with the formation of a new stereogenic center this was typically obtained as an almost equimolar mixture of two diastereomers and, accordingly, no attempt was made to distinguish the isomers.

The range of examples presented in Table 2, which were all conducted at room temperature with the exception of the carbohydrate-based example, displays the broad functional group compatibility of the method. A series of further experiments in which the reaction of cysteine derivative 25 with the methallyl seleno Bunte salt 42 and triphenylphosphine was conducted in methanol at room temperature in the presence of equimolar amounts of N-benzyloxycarbonyl tyrosine benzyl ester, N-benzyloxycarbonyl methionine, Nα-benzyloxycarbonyl lysine methyl ester, N-benzyloxycarbonyl arginine methyl ester, and N-benzyloxycarbonyl aspartic acid α-methyl ester, with no loss of yield and with recovery of the spectator amino acid, further confirmed the applicability of the reaction in the presence of most standard functional groups.

Unfortunately, all attempts at the preparation of tertiary allylic seleno Bunte salts or the equivalent selenocyanates, such as would be necessary for the introduction of a simple geranyl or farnesyl group to a sulfide, failed. This result is not surprising in view of the instability of secondary allylic selenocyanate and even phenylselenides noted by Sharpless.¹⁰⁶

Allylic Disulfide Rearrangement. The early work on the allylic disulfide rearrangement (Scheme 2)^{96, 101} indicated the considerable effect of substituents in the allylic moiety on reaction rate with the secondary and tertiary allylic disulfides undergoing rearrangement several orders of magnitude more rapidly than their primary counterparts in hot benzene, thereby providing grounds for hope that the rearrangement could be induced to function as required at room temperature with the correct substituent pattern. In addition, consideration of the mechanism of the allylic disulfide rearrangement leads to the hypothesis that the reaction should be facilitated in polar solvents capable of stabilizing the dipolar thiosulfoxide intermediate. We assign the dipolar structure to the thiosulfoxides, a class of compounds, which have not been isolated or characterized spectroscopically, by reasonable analogy with the sulfoxides. Thiosulfinates (ROS(S)OR) have been isolated and studied crystallographically, spectroscopically, and computationally, with the evidence pointing to the polar structure.¹¹⁸ Indeed, the early workers in the field noted a 6-fold to 10-fold increase in the rate of desulfurative rearrangement of 1,3-dimethylbut-2-enyl t-butyl disulfide with triphenylphosphine on going from benzene to ethanol/benzene (9/2) at 80° C.,⁹⁶ and Höle and Baldwin commented on the instability of methanolic solutions of 10 and 11 with respect to rearrangement, as compared to the neat samples.¹⁰¹

A series of secondary and tertiary allylic thiols were prepared by the [3,3]-sigmatropic rearrangement of a variety of primary allylic thiocarbonyl derivatives (Scheme 4, Table 3).¹¹⁹⁻¹²⁹ While this chemistry was straightforward, caution was necessary in the conversion of the rearrangement products to the thiols as these were found to undergo a known¹²¹ 1,3-shift to the isomeric primary allylic thiols under basic conditions. The optimum conditions for this cleavage involved the reduction of the thiol carbamates with lithium aluminum hydride,¹²¹ and the cleavage of the dithiocarbonates with ethanolamine. The thiols were then allowed to react with either 2,2′-dipyridyl disulfide, 2,2′-di(5-nitropyridyl) disulfide, or 2,2′-di(1,3-benzothiazolyl) disulfide^{1, 2, 75-77, 108, 130-133} for conversion to the corresponding allyl heteroaryl disulfides and ultimate transfer to the target thiols (Scheme 4, Table 3). It is noteworthy that sulfenylating agents derived from hindered allylic thiols have drawn little attention so far, with only a limited number of allylic benzothiazolyl disulfides being described in the literature.^{134, 135} In addition to these allylic heteroaryl disulfides, we also prepared a single example (75) of an S-allyl Se-phenyl selenosulfide, by reaction of the thiol with N-phenylselenophthalimide, as it had been reported that the selenosulfides were more effective at sulfenyl transfer than the corresponding disulfides.⁸⁰

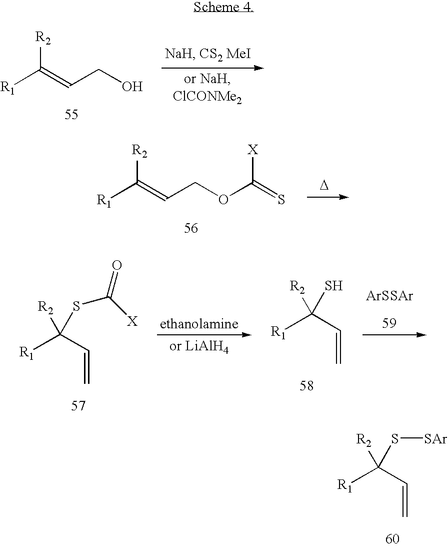

TABLE 3

| Allylic | Rearranged | | |
|---|---|---|---|
| Xanthates | Thiocarbamate | Thiol | Disulfide |
| (% yield) | (% yield) | (% yield) | (% yield) |
| 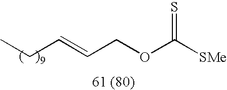 | 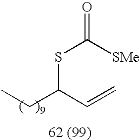 | 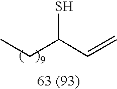 | 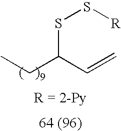 |
| | | | 4-NO₂-2-Py |
| | | | 65 (86) |
| | | | Bt |
| | | | 66 (81) |
| 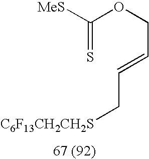 | 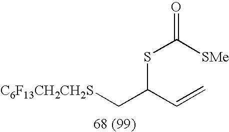 | 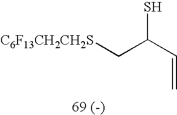 | 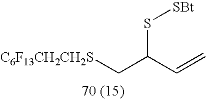 |
| 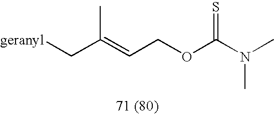 | 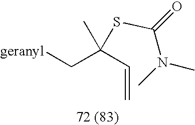 | 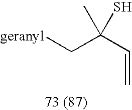 | 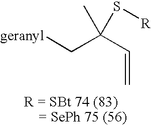 |
| 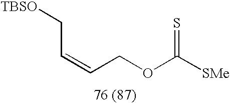 | 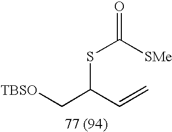 | 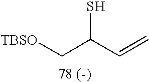 | 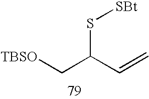 |

These allylic heteroaryl disulfides exhibited smooth sulfenyl transfer to a selection of primary thiols in either benzene or methanol/acetonitrile mixtures at room temperature (Table 4). Addition of either triphenylphosphine or (4-dimethylaminophenyl)diphenylphosphine then promoted the desired desulfurative allylic rearrangement. For those reactions that were carried out in benzene, the sulfenyl transfer was affected at room temperature, after which the phosphine was added and the reaction mixture heated to reflux to provoke the rearrangement. On the other hand, when reactions were conducted in methanol and acetonitrile no heating was required for the entire sequence, which is consistent with mechanistic considerations. With the more highly substituted allyl heteroaryl disulfides, sulfenyl transfer was relatively slow, but was accelerated very significantly by the addition of triethylamine. Interestingly, sulfenyl transfer from the selenosulfide 75 was also slow, but was accelerated in the presence of triethylamine. The accelerated reactions reported in the literature for sulfenyl transfer from selenosulfides, as compared to disulfides, also appear to be the result of the inclusion of triethylamine.⁸⁰ No obvious consequence in terms of reactivity was observed when the sulfenyl transfer group was changed from 5-nitro-2-pyridyl, to 2-pyridyl, to 2-benzothiazolyl, but the differing polarities of the corresponding heteroaryl sulfides formed as byproducts can be used to advantage in the case of otherwise difficult purifications. Similarly, no significant difference in the rate of the desulfurative rearrangement was observed on changing from triphenylphosphine to (4-dimethylaminophenyl)diphenylphosphine 84, but the latter did afford a practical advantage in terms of purification of the final products.

The desulfurative rearrangement of the secondary allylic disulfides takes place with high E-selectivity as anticipated on the basis of related [2,3]-sigmatropic rearrangements such as the Evans-Mislow rearrangement¹⁰³ or the [2,3]-Wittig rearrangement.¹³⁶⁻¹³⁸ With the tertiary allylic disulfides, E/Z mixtures of the rearranged products are formed that slightly favor the E-isomer. Interestingly, in the case of the nerolidyl selenosulfide 75 the product 86 was obtained as a more complex mixture of isomers, presumably due to isomerization of the central alkene in the resulting farnesyl chain by the selenide byproducts in combination with light.¹³⁹ When the reaction of the cysteine derivative 83 with the sulfenyl transfer agent 66 and triphenylphosphine was carried out in a mixture of deuteriomethanol and deuterioacetonitrile no incorporation of deuterium at the amino acid α-center was observed, indicating that the reaction conditions do not provoke racemization.

The examples presented in Table 4 attest to the broad functional group compatibility of the formation and desulfurative rearrangement of secondary and tertiary allylic disulfides and draw attention to the complementary nature of the process with the deselenative allylic selenosulfide protocol, with all classes of primary, secondary, and tertiary allylic sulfides accessible by one of the two reaction sequences at room temperature and without the need for electrophilic reagents.

TABLE 4

| | Sulfenating | | | | Product |
|---|---|---|---|---|---|
| Thiol | agent | Solv. | PR₃ | T ° C. | (% yield) |
| 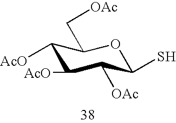 | 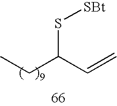 | C₆H₆ | PPh₃ | reflux | 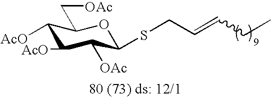 |
| 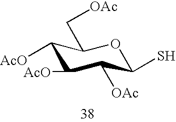 | 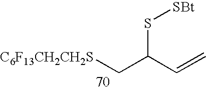 | C₆H₆ | PPh₃ | reflux | 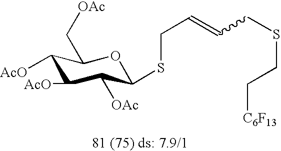 |
| 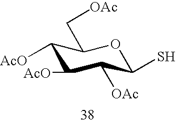 | 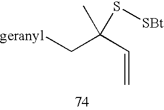 | C₆H₆ | PPh₃ | reflux | 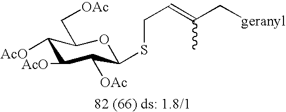 |
| 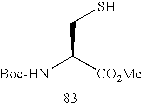 | 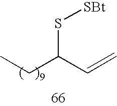 | A | 84 | RT | 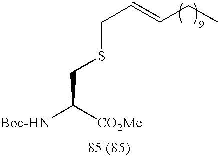 |
| 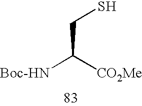 | 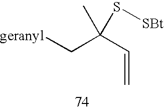 | A | PPh₃ | RT | 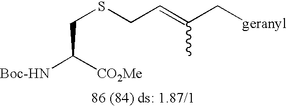 |
| 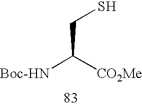 | 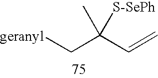 | A | PPh₃ | RT | 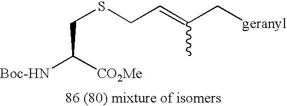 |
| 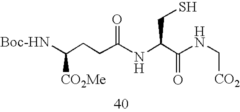 | 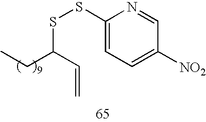 | A | 84 | RT | 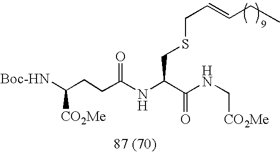 |
| 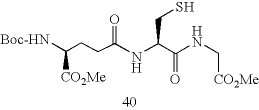 | 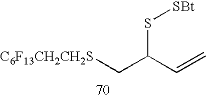 | A | PPh₃ | RT | 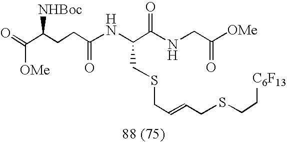 |
| 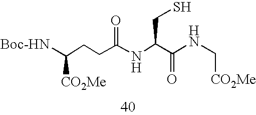 | 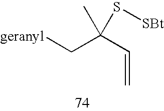 | A | PPh₃ | RT | 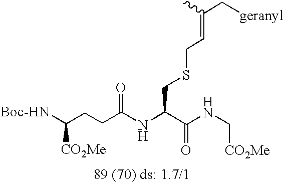 |
| 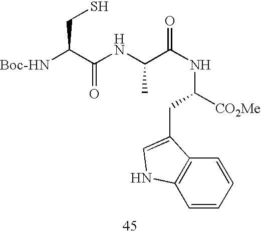 | 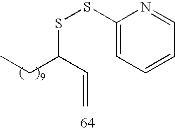 | A | PPh₃ | RT | 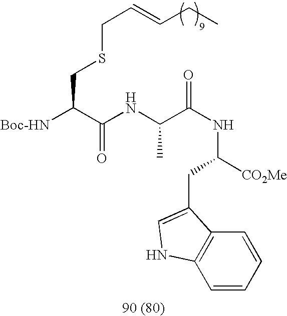 |
| 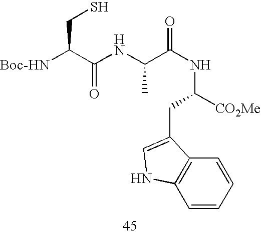 | 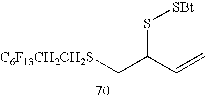 | A | PPh₃ | RT | 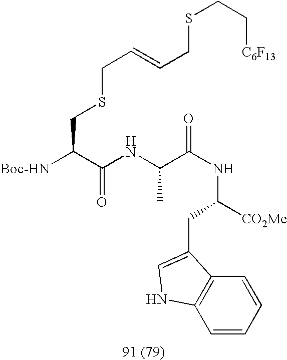 |
| 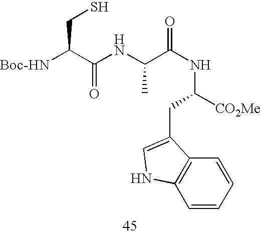 | 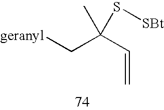 | A | PPh₃ | RT | 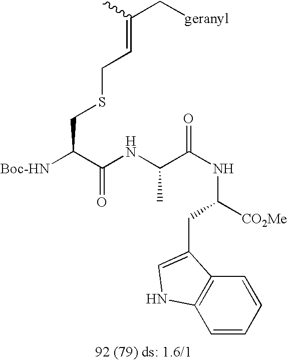 |

In Table 4, Solvent A is MeOH/MeCN (1:1);
Phosphine (PR₃) 84 is (4-Me₂N—C₆H₄)diphenylphosphine.

Use in Aqueous Media. The applicability of the desulfurative allylic rearrangement in aqueous media was first probed with glutathione 93 and sulfenylating agent 64. Disulfide 64 was reacted with glutathione 93 in a 2/1/1 mixture of Tris Buffer/CH₃CN/THF, with a substrate concentration of about 0.02 M at room temperature, followed by addition of triphenylphosphine, which smoothly yielded the lipidated glutathione 94 in 70% yield (Scheme 5).

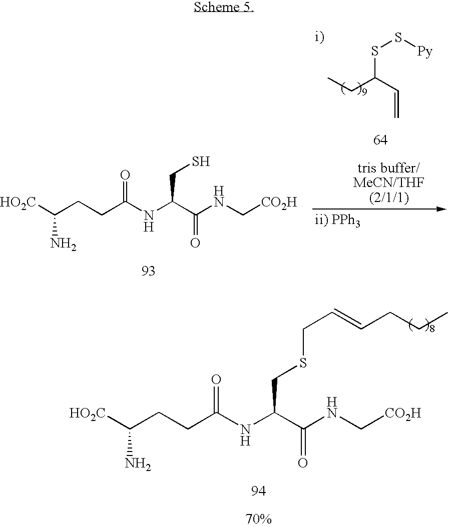

The chemoselectivity of the rearrangement was further explored with the commercial decapeptide fibronectin fragment 95.^{140, 141} This decapeptide was selected as a proving ground due to the dense array of the more challenging amino acid side chains that it presents. As with glutathione 93, sulfenylation of the cysteine moiety of the decapeptide 95, followed by phosphine-promoted rearrangement, was accomplished at room temperature in aqueous buffer, with the aid of organic co-solvents, and resulted in the isolation of the lipidated decapeptide 96 in 70% yield (Scheme 6). The location of the tridecene chain on the cysteine group in 96 was established by tandem MS/MS experiments.

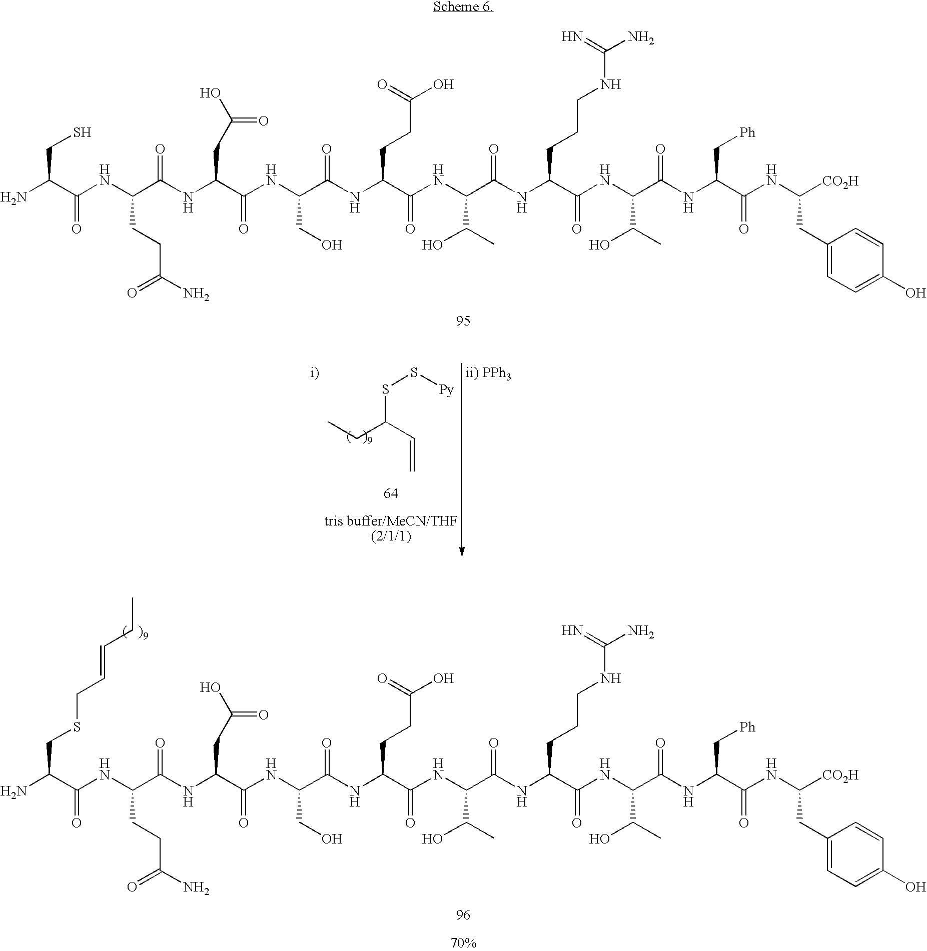

As a final demonstration of the protocol in aqueous media we prepared an octapeptide 101 in which the cysteine residue was located four residues from a C-terminal methionine unit, so as to mimic the Cys-AA¹-AA²-Met sequence targeted by the farnesyl transferase enzymes.¹⁴²⁻¹⁴⁴ The other amino acids in this synthetic peptide were selected so as to provide a measure of steric hindrance to the cysteine moiety. Two tetrapeptides were assembled by routine methods,^{145, 146} and 97, which was destined to become the N-terminal fragment of the target, was saponified, converted to the ethylthio ester 98 with HATU and ethanethiol,^{147, 148} and finally released from the carbamate protecting group in the standard manner (Scheme 7). This sequence resulted in an inseparable mixture of two diastereomers 99 at the C-terminal alanine, a problem known to plague thioester synthesis for native chemical ligation. The use of the PyBop activation method for thiol ester formation, as applied recently by the Kajihara group, appears to hold promise for the non-racemizing synthesis of C-terminal thiol esters, but no attempt was made to apply it in the present synthesis.¹⁴⁹ Coupling of thioester 99 with tetrapeptide 100 under conditions of native chemical ligation afforded the target octapeptide 101 in 51% yield as a 1.3/1 mixture of two diastereomers, which was separated by preparative RP-HPLC. The major diastereoisomer, presumed to be the all L-octapeptide, was then allowed to react with the nerolidyl benzothiazolyl disulfide 74 under buffered aqueous conditions at room temperature, followed by addition of triphenylphosphine leading, overall, to the farnesylated octapeptide 102 in 66% isolated yield (Scheme 7). This farnesylation reaction occurred with the formation of E/Z-isomers mixtures in approximately equal ratio in line with the model experiments in organic solution (Table 4).

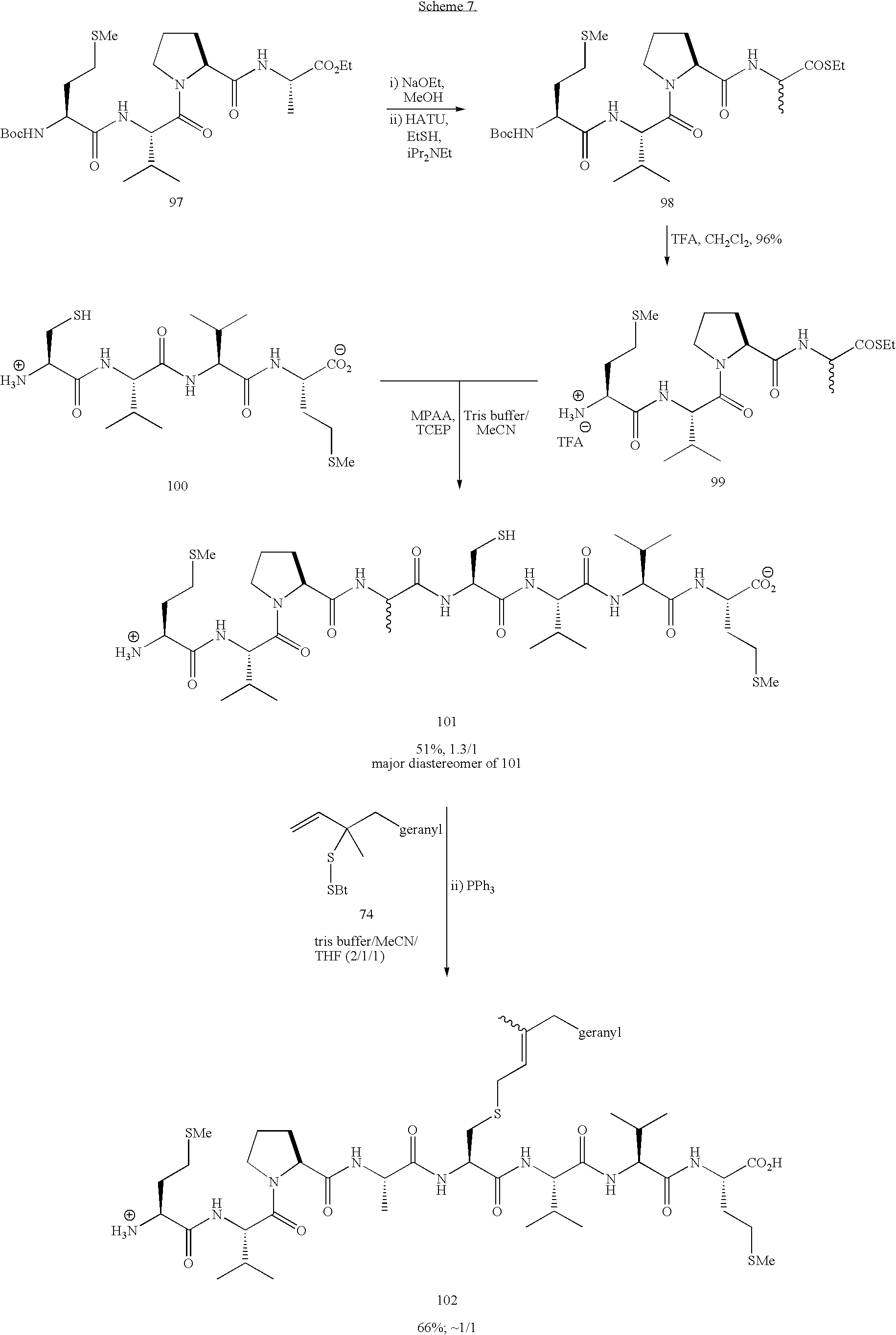

The examples of Schemes 5-7 are distinct from other recent functionalizations of sulfur in cysteine containing peptides⁵² as they require neither the use of electrophilic reagents,^{1, 2, 82} nor the incorporation of modified amino acid residues into the peptide backbone prior to functionalization,⁸³⁻⁹⁰ nor the synthesis of peptides with previously functionalized amino acid building blocks.⁹¹⁻⁹³ These factors combine to render this chemistry both highly chemoselective, and highly efficient in terms of steps required on the actual peptide target. In contrast to other methods involving the addition of nucleophilic thiols to dehydroalanine groups inserted into peptides,⁸³ the products are obtained as single diastereomers with the sole exception being the formation of E/Z-mixtures when the allylic sulfide contains a trisubstituted alkene.

Rearrangement in the Absence of Phosphines. We have noted that the attempted purification of the cysteine derived disulfide 103 by chromatography over silica gel resulted in the isolation of 13% of the allylically rearranged thioether 85 along with the disulfide itself (Scheme 8).⁹⁵ On standing in deuteriochloroform that had not been treated to remove acidic impurities the same disulfide 103 underwent partial rearrangement to 85 with loss of sulfur.⁹⁵ In addition, morpholine or piperidine have been reported to drive the related allylic sulfoxide/sulfenate rearrangement.¹⁰³ We have now found that the allylic disulfide rearrangement can be efficiently performed in the absence of a phosphine.

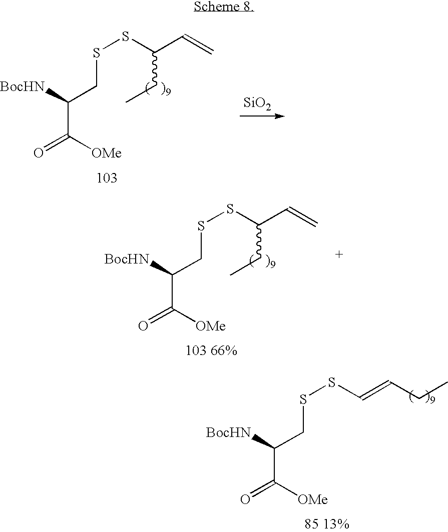

The apparent acid-catalyzed rearrangement of 103 to 85 with loss of sulfur did not provide a satisfactory general method for the rearrangement. However, after some investigation, we have found that the activation energy necessary to facilitate the rearrangement can be achieved, with no added phosphine, by stirring the disulfide with two equivalents of an amine catalyst (piperidine) at room temperature in a solvent such as methanol or simply by heating to reflux in the solvent, with or without a catalyst. Examples of rearrangements so-conducted are presented in Table 5 using a sulfenyl transfer reagent 104 derived by removal of the silyl group from sulfenyl donor 79. Appropriate control experiments conducted with triphenylphosphine at room temperature are also reported in Table 5.

TABLE 5

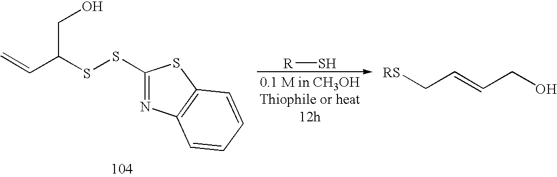

| | | | PPh₃ | Piperidine | Heat |
|---|---|---|---|---|---|
| | Thiol | | % Yield | % Yield | % Yield |
| Entry | RSH | Product | E/Z ratio | E/Z ratio | E/Z ratio |
| 1 | 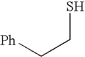 | 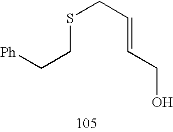 | 45 E only | 62 2.6:1 | 78 E only |
| 2 | 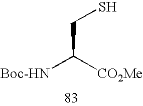 | 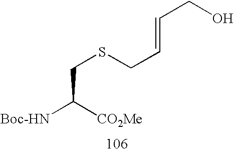 | — | — | 67 E only |
| 3 | 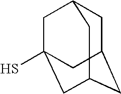 | 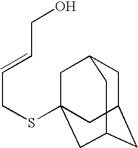 | — | — | 68 E only |
| 4 | CH₃(CH₂)₇SH | 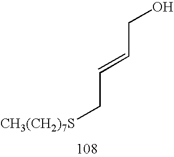 | — | — | 75 E only |
| 5 | 4-Me-Ar—SH | 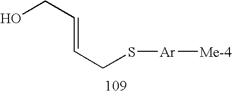 | 18 E only | 66 2.5:1 | 66 E only |
| 6 | PhSH | 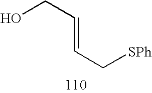 | — | — | 63 E only |
| 7 | 4-Cl-Ar—SH | 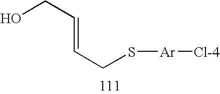 | — | — | 62 E only |
| 8 | 4-NO₂-Ar—SH | 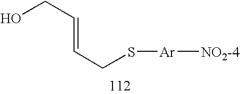 | — | — | 20 E only |
| 9 | 2-Py—SH | 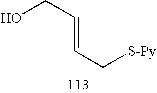 | — | — | 45 E only |
| 10 | 2-HO—Ar—SH | 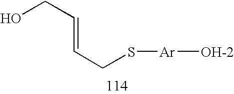 | — | — | 57 E only |

Both the piperidine and the refluxing methanol conditions for the first time enable the efficient desulfurative rearrangement of ally aryl disulfides in the absence of a phosphine, thereby opening up new avenues for the synthesis of ally aryl sulfides and extending the overall scope of the reaction. The rearrangements of the allyl aryl disulfides presented in Table 5, and conducted in the absence of phosphine, but with the aid of either piperidine or hot methanol, are to be contrasted with the attempted promotion of such a rearrangement set out in Table 1, when the excision of sulfur was observed without the allylic rearrangement. This process can also be applied to a tertiary thiol. In addition, no racemization was observed in the case of the cysteine derivative, as determined in the usual manner by the use of deuteriomethanol as solvent.

Conclusions. The deselenative allylic rearrangement of primary allyl selenosulfides and the desulfurative rearrangement of secondary and tertiary allylic are powerful and complementary techniques for the synthesis of primary, secondary, and tertiary allylic sulfides at room temperature. The preparation of the selenosulfides and disulfides requires no electrophilic reagent, leading to highly chemoselective methods for the permanent modification of thiols. The chemoselectivity of the two reactions enables their application to unprotected peptides in aqueous media in the presence of all types of standard amino acid side chains, thereby providing a powerful means for the direct and permanent modification of cysteine containing peptides.

The use of the terms “a” and “an” and “the” and similar references in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

(1) Hermanson, G. T. Bioconjugate Techniques; Academic Press: San Diego, 1996.
(2) Lundblad, R. L. Chemical Reagents for Protein Modification; 3 ed.; CRC Press: Boca Raton, 2005.
(3) van Swieten, P. F.; Leeuwenburgh, M. F.; Kessler, B. M.; Overkleeft, H. S. Org. Biomol. Chem. 2005, 3, 20-27.
(4) Peri, F.; Nicotra, F. Chem Comm 2004, 623-627.
(5) Pratt, M. R.; Bertozzi, C. R. Chem. Soc. Rev 2005, 34, 58-68.
(6) Prescher, J. A.; Dube, D. H.; Bertozzi, C. R. Nature 2004, 430, 873-877.
(7) Hang, H. C.; Bertozzi, C. R. Acc. Chem. Res. 2001, 34, 727-736.
(8) Nilsson, B. L.; Hondal, R. J.; Soellner, M. B.; Raines, R. T. J. Am. Chem. Soc. 2003, 125, 5268-5269.
(9) Bode, J. W. Curr. Op. Drug Disc. Dev. 2006, 9, 765-775.
(10) Nilsson, B. L.; Soellner, M. B.; Raines, R. T. Ann. Rev. Biophys. Biomol. Structure 2005, 34, 91-118.
(11) Kalia, J.; Raines, R. T. ChemBioChem 2006, 7, 1376-1383.
(12) Lin, H.; Walsh, C. T. J. Am. Chem. Soc. 2004, 126, 13998-14003.
(13) Langenhan, J. M.; Thorson, J. S. Current Organic Synthesis 2005, 2, 59-81.
(14) Agard, N. J.; Baskin, J. M.; Prescher, J. A.; Lo, A.; Bertozzi, C. R. ACS Chem. Biol. 2006, 1,644-648.
(15) Köhn, M.; Breinbauer, R. Angew. Chem. Int. Ed. 2004, 43, 3106-3116.
(16) Lin, F. L.; Hoyt, H. M.; van Halbeek, H.; Bergman, R. G.; Bertozzi, C. R. J. Am. Chem. Soc. 2005, 127, 2686-2695.
(17) Grandjean, C.; Boutonnier, A.; Guerreiro, C.; Fournier, J.-M.; Mulard, L. A. J. Org. Chem. 2005, 70, 7123-7132.
(18) Hang, H. C.; Yu, C.; Pratt, M. R.; Bertozzi, C. R. J. Am. Chem. Soc. 2004, 126, 6-7.
(19) Lemieux, G. A.; de Graffenreid, C. L.; Bertozzi, C. R. J. Am. Chem. Soc. 2003, 125, 4708-4709.
(20) Saxon, E.; Luchansky, S. J.; Hang, H. C.; Yu, C.; Lee, S. C.; Bertozzi, C. R. J. Am. Chem. Soc. 2002, 124, 14893-14902.
(21) Saxon, E.; Bertozzi, C. R. Science 2000, 287, 2007-2010.
(22) Saxon, E.; Armstrong, J. I.; Bertozzi, C. R. Org. Lett. 2000, 2, 2141-2143.
(23) Bianchi, A.; Bernardi, A. J. Org. Chem. 2006, 71, 4565-4577.
(24) Kim, H.; Cho, J. K.; Aimoto, S.; Lee, Y.-S. Org. Lett. 2006, 8, 1149-1151.
(25) Soellner, M. B.; Nilsson, B. L.; Raines, R. T. J. Am. Chem. Soc. 2006, 128, 8820-8828.
(26) Liu, L.; Hong, Z.-Y.; Wong, C.-H. ChemBioChem 2006, 7, 429-432.
(27) Soellner, M. B.; Tam, A.; Raines, R. T. J. Org. Chem. 2006, 71, 9824-9830.
(28) Doores, K. J.; Mimura, Y.; Dwek, R. A.; Rudd, P. M.; Elliott, T.; Davis, B. G. Chem. Commun. 2006, 1401-1403.
(29) Kolb, H. C.; Finn, M. G.; Sharpless, K. B. Angew. Chem. Int. Ed. 2001, 40, 2004-2021.
(30) Lutz, J.-F. Angew. Chem. Int. Ed. 2007, 46, 1018-1025.
(31) Breinbauer, R.; Köhn, M. ChemBioChem 2003, 4, 1147-1149.
(32) Manetsch, R.; Krasinski, A.; Radic, Z.; Raushel, J.; Taylor, P.; Sharpless, K. B.; Kolb, H. C. J. Am. Chem. Soc. 2004, 126, 12809-12818.
(33) Devaraj, N. K.; Miller, G. P.; Ebina, W.; Kakaradov, B.; Collman, J. P.; Kool, E. T.; Chidsey, C. E. D. J. Am. Chem. Soc. 2005, 127, 8600-8601.
(34) Whiting, M.; Muldoon, J.; Lin, Y.-C.; Silverman, S. M.; Lindstrom, W.; Olson, A. J.; Kolb, H. C.; Finn, M. G.; Sharpless, K. B.; Elder, J. H.; Fokin, V. V. Angew. Chem. Int. Ed. 2006, 45, 1435-1439.
(35) Srinivasan, R.; Uttamchandani, M.; Yao, S. Q. Org. Lett. 2006, 8, 713-716.
(36) Chittaboina, S.; Xie, F.; Wang, Q. Tetrahedron Lett. 2005, 46, 2331-2336.
(37) Hotha, S.; Kashyap, S. J. Org. Chem. 2006, 71, 364-367.
(38) Gauchet, C.; Labadie, G. R.; Poulter, C. D. J. Am. Chem. Soc. 2006, 128, 9274-9275.
(39) Wan, Q.; Chen, J.; Chen, G.; Danishefsky, S. J. J. Org. Chem. 2006, 71, 8244-8249.
(40) van der Peet, P.; Gannon, C. T.; Walker, I.; Dinev, Z.; Angelin, M.; Tam, S.; Ralton, J. E.; McConville, M. J.; Williams, S. J. ChemBioChem 2006, 7, 1384-1391.
(41) Shangguan, N.; Katukojvala, S.; Greenberg, R.; Williams, L. J. J. Am. Chem. Soc. 2003, 125, 7754-7755.
(42) Merkx, R.; Brouwer, A. R.; Rijkers, D. T. S.; Liskamp, R. M. J. Org. Lett. 2005, 7, 1125-1128.
(43) Kolakowski, R. V.; Shangguan, N.; Sauers, R. R.; Williams, L. J. J. Am. Chem. Soc. 2006, 128, 5695-5702.
(44) Knapp, S.; Darout, E. Org. Lett. 2005, 7, 203-206.
(45) Kolakowski, R. V.; Shangguan, N.; Williams, L. J. Tetrahedron Lett. 2006, 47, 1163-1166.
(46) Barlett, K. N.; Kolakowski, R. V.; Katukojvala, S.; Williams, L. J. Org. Lett. 2006, 8, 823-826.
(47) Wu, X.; Hu, L. J. Org. Chem. 2007, 72, 765-774.
(48) Dirksen, A.; Hackeng, T. M.; Dawson, P. E. Angew. Chem. Int. Ed. 2006, 45, 7581-7584.
(49) Liu, H.; Wang, L.; Brock, A.; Wong, C.-H.; Schultz, P. G. J. Am. Chem. Soc. 2003, 125, 1702-1703.
(50) Pearce, 0. M. T.; Fisher, K. D.; Humphries, J.; Seymour, L. W.; Smith, A.; Davis, B. G. Angew. Chem. Int. Ed. 2005, 44, 1057-1061.
(51) Kubler-Kielb, J.; Pozsgay, V. J. Org. Chem. 2005, 70, 6987-6990.
(52) Pachamuthu, K.; Schmidt, R. R. Chem. Rev. 2006, 106, 160-187.
(53) Muir, T. W.; Sondhi, D.; Cole, P. A. Proc. Natl. Acad. Sci. USA 1998, 95, 6705.
(54) Noren, C. J.; Wang, J. M.; Perler, F. B. Angew. Chem. Int. Ed. 2000, 39, 450.
(55) Muralidharan, V.; Muir, T. W. Nat. Methods 2006, 3, 429.
(56) Dawson, P. E.; Muir, T. W.; Clark-Lewis, I.; Kent, S. B. H. Science 1994, 266, 776-779.
(57) Kochendoerfer, G. G.; Kent, S. B. H. Curr. Opinion. Chem. Biol. 1999, 3, 665-671.
(58) Dawson, P. E.; Kent, S. B. H. Ann. Rev. Biochem. 2000, 69, 923-960.
(59) Coltart, D. M. Tetrahedron 2000, 56, 3449-3491.
(60) Yeo, D. S. Y.; Srinivasan, R.; Chen, G. Y. J.; Yao, S. Q. Chem. Eur. J. 2004, 10, 4664-4672.
(61) Bang, D.; Kent, S. B. H. Proc. Natl. Acad. Sci., U.S.A. 2005, 102, 5014-5019.
(62) Bang, D.; Kent, S. B. H. Angew. Chem. Int. Ed. 2004, 43, 2534-2538.
(63) Dose, C.; Seitz, O. Org. Lett. 2005, 7, 4365-4368.
(64) Johnson, E. C. B.; Durek, T.; Kent, S. B. H. Angew. Chem. Int. Ed. 2006, 45, 3283-3287.
(65) Johnson, E. C. B.; Kent, S. B. H. J. Am. Chem. Soc. 2006, 128, 6640-6646.
(66) Brik, A.; Yang, Y.-Y.; Ficht, S.; Wong, C.-H. J. Am. Chem. Soc. 2006, 128, 5626-5627.
(67) Brik, A.; Ficht, S.; Yang, Y.-Y.; Bennett, C. S.; Wong, C.-H. J. Am. Chem. Soc. 2006, 128, 15026-15033.
(68) Yan, L. Z.; Dawson, P. E. J. Am. Chem. Soc. 2001, 123, 526-533.
(69) Hondal, R. J.; Nilsson, B. L.; Raines, R. T. J. Am. Chem. Soc. 2001, 123, 5140-5141.
(70) Botti, P.; Carrasco, M. R.; Kent, S. B. H. Tetrahedron Lett. 2001, 42, 1831-1833.
(71) Clive, D. L. J.; Hisaindee, S.; Coltart, D. M. J. Org. Chem. 2003, 68, 9247-9254.
(72) Tchertchian, S.; Hartley, O.; Botti, P. J. Org. Chem. 2004, 69, 9208-9214.
(73) Chen, G.; Warren, D. J.; Chen, J.; Wu, B.; Wan, Q.; Danishefsky, S. J. J. Am. Chem. Soc. 2006, 128, 7460-7462.
(74) Wu, B.; Chen, J.; Warren, J. D.; Chen, G.; Hua, Z.; Danishefsky, S. J. Angew. Chem. Int. Ed. 2006, 45, 4116-4125.
(75) Kenyon, G. L.; Bruice, T. W. Methods Enzym. 1977, 47, 407-430.
(76) Wynn, R.; Richards, F. M. Methods Enzym. 1995, 251, 351-356.
(77) Faulstich, H.; Heintz, D. Methods Enzym. 1995, 251, 357-366.
(78) Macindoe, W. M.; van Oijen, A. H.; Boons, G.-J. Chem. Commun. 1998, 847-848.
(79) Grayson, E. J.; Ward, S. J.; Hall, A. J.; Rendle, P. M.; Gamblin, D. P.; Batsanov, A. S.; Davis, B. G. J. Org. Chem. 2005, 70, 9740-9754.
(80) Gamblin, D. P.; Garnier, P.; van Kasteren, S.; Oldham, N. J.; Fairbanks, A. J.; Davis, B. G. Angew. Chem. Int. Ed. 2004, 43, 828-833.
(81) Gamblin, D. P.; Garnier, S. J.; Ward, N. J.; Oldham, A. J.; Fairbanks, A. J.; Davis, B. G. Org. Biomol. Chem. 2003, 1, 3642-3644.
(82) Ludolph, B.; Eisele, F.; Waldmann, H. J. Am. Chem. Soc. 2002, 124, 5954-5955.
(83) Zhu, Y.; van der Donk, W. A. Org. Lett. 2001, 3, 1189-1192.
(84) Thayer, D. A.; Yu, H. N.; Galan, M. C.; Wong, C.-H. Angew. Chem. Int. Ed. 2005, 44, 4596-4599.
(85) Jobron, L.; Hummel, G. Org. Lett. 2000, 2, 2265-2267.
(86) Pachamuthu, K.; Zhu, X.; Schmidt, R. R. J. Org. Chem. 2005, 70, 3720-3723.
(87) Cohen, S. B.; Halcomb, R. L. Org. Lett. 2001, 3, 405-407.
(88) Cohen, S. B.; Halcomb, R. L. J. Am. Chem. Soc. 2002, 124, 2534-2543.
(89) Galonic, D. P.; van der Donk, W.; Gin, D. Y. J. Am. Chem. Soc. 2004, 126, 12712-12713.
(90) Galonic, D. P.; Ide, N. D.; van der Donk, W. A.; Gin, D. Y. J. Am. Chem. Soc. 2005, 127, 7359-7369.
(91) Palomo, J. M.; Lumbierres, M.; Waldmann, H. Angew. Chem. Int. Ed. 2006, 45, 477-481.
(92) Durek, T.; Alexandrov, K.; Goody, R. S.; Hildebrand, A.; Heinemann, I.; Waldmann, H. J. Am. Chem. Soc. 2004, 126, 16368-16378.
(93) Kragol, G.; Lumbierres, M.; Palomo, J. M.; Waldmann, H. Angew. Chem. Int. Ed. 2004, 43, 5839-5842.
(94) Crich, D.; Krishnamurthy, V.; Hutton, T. K. J. Am. Chem. Soc. 2006, 128, 2544-2545.
(95) Crich, D.; Brebion, F.; Krishnamurthy, V. Org. Lett. 2006, 8, 3593-3596.
(96) Moore, C. G.; Trego, G. R. Tetrahedron 1962, 18, 205-218.
(97) Evans, M. B.; Higgins, G. M. C.; Moore, C. G.; Porter, M.; Saville, B.; Smith, J. F.; Trego, B. R.; Watson, A. A. Chem. & Ind. 1960, 897.
(98) Blackburn, G. M.; 011 is, W. D. J. Chem. Soc., Chem. Commun. 1968, 1261-1262.
(99) Barnard, D.; Houseman, T. H.; Porter, M.; Tidd, B. K. J. Chem. Soc., Chem. Commun. 1969, 371-372.
(100) Pilgram, K.; Phillips, D. D.; Korte, F. J. Org. Chem. 1964, 29, 1844-1847.
(101) Höfle, G.; Baldwin, J. E. J. Am. Chem. Soc. 1971, 93, 6307-6308.
(102) Kutney, G. W.; Turnbull, K. Chem. Rev. 1982, 82, 333-357.
(103) Evans, D. A.; Andrews, G. C. Acc. Chem. Res. 1974, 7, 147-155.
(104) Baechler, R. D.; Hummel, J. P.; Mislow, K. J. Am. Chem. Soc. 1973, 95, 4442-4444.
(105) Block, E.; Iyer, R.; Grisoni, S.; Saha, C.; Belman, S.; Lossing, F. P. J. Am. Chem. Soc. 1988, 110, 7813-7827.
(106) Sharpless, K. B.; Lauer, K. B. J. Org. Chem. 1972, 37, 3973-3974.
(107) Riague, E. H.; Guillemin, J.-C. Organometallics 2002, 21, 68-73.
(108) Stoner, E. J.; Negrón, G.; Gaviño, R. In Handbook of Reagents for Organic Synthesis: Reagents for Glycoside, Nucleotide, and Peptide Synthesis; Crich, D., Ed.; Wiley: Chichester, 2005, pp 330-338.
(109) Klayman, D. L.; White, J. D.; Sweeney, T. R. J. Org. Chem. 1964, 29, 3737-3738.
(110) Milligan, B.; Swan, J. M. J. Chem. Soc. 1963, 6008-****.
(111) Harpp, D. N.; Gleason, J. G. J. Am. Chem. Soc. 1971, 93, 2437-2445.
(112) Patarasalculchai, N.; Southwell-Keely, P. T. Phosphorus and Sulfur 1985, 22, 277-282.
(113) Klayman, D. L.; Günther, W. H. H., Eds. Organic selenium compounds: their chemistry and biology; Wiley Interscience: New York, 1973.
(114) Clark, E. R.; Al-Turaihi, M. A. S. J. Organomet. Chem. 1977, 134, 181-187.
(115) Guillemin, J. C., Bouayad, A., Vijaykumar D. ChemComm 2000, 13, 1163-1164.
(116) Emerson, D. W.; Booth, J. K. J. Org. Chem. 1965, 30, 2480-2481.
(117) Ferrier, R. J.; Vethaviyaser, N. J. Chem. Soc. (C) 1971, 1907-1913.
(118) Zysman-Colman, E.; Nevins, N.; Eghbali, N.; Snyder, J. P.; Harpp, D. N. J. Am. Chem. Soc. 2005, 128, 291-304.
(119) Garmaise, D. l.; Uchiyama, A.; McKay, A. F. J. Org. Chem. 1962, 27, 4509-4512.
(120) Ferrier, R. J.; Vethaviyasar, N. J. Chem. Soc., Chem. Commun. 1970, 1385-1387.
(121) Hackler, R. E.; Balko, T. W. J. Org. Chem. 1973, 38, 2106-2110.
(122) Harano, K.; Taguchi, T. Chem. Pharm. Bull. 1975, 23, 467-472.
(123) Harano, K.; Taguchi, T. Chem. Pharm. Bull. 1972, 20, 2348-2356.
(124) Harano, K.; Taguchi, T. Chem. Pharm. Bull. 1972, 20, 2357-2365.
(125) Nakai, T.; Mimura, T.; Ari-Izumi, A. Tetrahedron Lett. 1977, 2425-2428.
(126) Ueno, Y.; Sano, H.; Okawara, M. Tetrahedron Lett. 1980, 21, 1767-1770.
(127) Eto, M.; Tajiri, O.; H., N.; K., H. Tetrahedron 1998, 54, 8009-8014.
(128) Nakai, T.; Ari-Izumi, A. Tetrahedron Lett. 1976, 17, 2335-2338.
(129) Harano, K.; Ohizumi, N.; Hisano, T. Tetrahedron Lett. 1985, 26, 4203-4206.
(130) Talgoy, M. M.; Bell, A. W.; Duckworth, H. W. Can. J. Biochem. 1979, 57, 822-833.
(131) Kimura, T.; Matsueda, R.; Nakagawa, Y.; Kaiser, E. T. Anal. Biochem. 1982, 122, 274-282.
(132) Brzezinska, E.; Ternay, A. L. J. Org. Chem. 1994, 59, 8239-8244.
(133) Rabanal, F.; DeGrado, W. F.; Dutton, P. L. Tetrahedron Lett. 1996, 37, 1347-1350.
(134) Watson, A. A. J. Chem. Soc. (C) 1964, 2100-2107.
(135) Morrison, N. J. J. Chem. Soc., Perkin Trans. 11984, 101-106.
(136) Nakai, T.; Mikami, K. Chem. Rev. 1986, 86, 885-902.
(137) Nakai, T.; Mikami, K. In Org. React.; Paquette, L. A., Ed.; John Wiley & Sons: New York, 1994; Vol. 46, pp 105-209.
(138) Mikami, K.; Nakai, T. Synthesis 1991, 594-604.
(139) Barrett, A. G. M.; Barton, D. H. R.; Johnson, G.; Nagubandi, S. Synthesis 1978, 741.
(140) Boucaut, J.-C.; Darribère, T.; Poole, T. J.; Aoyama, H.; Yamada, K. M.; Thierry, J. P. J. Cell. Biol. 1984, 99, 1822-1830.
(141) Yamada, K. M.; Kennedy, D. W. J. Cell. Biol. 1984, 99, 29-36.
(142) Casey, P. J. Science 1995, 266, 221-.
(143) Kale, T. A.; Hsieh, S.-a. J.; W., R. M.; Distefano, M. D. Curr. Top. Med. Chem. 2003, 3, 1043-1074.
(144) Brunsveld, L.; Kuhlmann, J.; Alexandrov, K.; Wittinghofer, A.; Goody, R. S.; Waldmann, H. Angew. Chem. Int. Ed. 2006, 45, 6622-6646.
(145) Bodanszky, M.; Bodanszky, A. The Practice of Peptide Synthesis; 2nd ed.; Springer-Verlag: Heidelberg, 1994.
(146) Goodman, M.; Mahwah, A. F.; Moroder, L.; Toniolo, C., Eds. Houben-Weyl. Methods in Organic Chemistry. Synthesis of Peptides and Peptidomimetics; Thieme: Stuttgart, 2002-2003; Vol. E22a-e.
(147) Schnolzer, M.; Alewood, P.; Jones, A.; Alewood, D.; Kent, S. B. H. Int. J. Peptide Protein Res. 1992, 40, 180-193.
(148) Clippingdale, A. B.; Barrow, C. J.; Wade, J. D. J. Pept. Sci. 2000, 6, 225.
(149) Kajihara, Y.; Yoshihara, A.; Hirano, K.; Yamamoto, N. Carbohydr. Res. 2006, 341, 1333-1340.

1. A dechalcogenative method for the preparation of an allylic sulfide comprising contacting an activated chalcogenide of Formula (I) with a thiol of Formula (II) for a period of time sufficient to form an intermediate of Formula (III), and supplying sufficient activation energy to the intermediate of Formula (III), in a suitable solvent, to induce a [2,3]-sigmatropic rearrangement therein to form an allylic sulfide of Formula (IV), with concomitant loss of chalcogen Z, as set forth in the following reaction scheme: 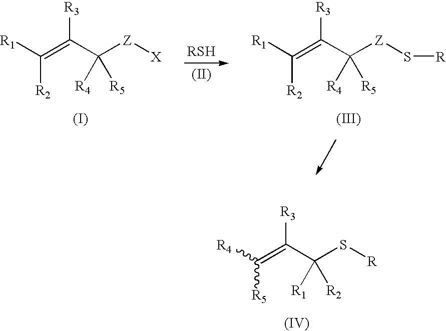
wherein X is an activating group selected from the group consisting of CN, S-pyridyl, S-heteroaryl, SO₂-aryl, and SO₃Y; Y is an alkali metal ion; Z is Se or S; R¹, R², R³, R⁴, and R⁵ are each independently 14 or a hydrocarbon moiety; and R is an organic moiety.

2. The method of claim 1 wherein the organic moiety, R, comprises an alkyl group, a substituted-alkyl group, an aryl group, an S-heteroaryl group, a substituted-aryl group, an amino acid, a carbohydrate, a nucleic acid, a peptide nucleic acid, a peptide, or a group consisting of two or more of the foregoing bound together.

3. The method of claim 1 wherein Z is S and X is S-pyridyl.

4. The method of claim 1 wherein Z is S and X is S-2-benzothiazolyl.

5. The method of claim 1 wherein Z is Se and X is CN.

6. The method of claim 1 wherein Z is Se and X is SO₃Y wherein Y is Na or K.

7. The method of claim 1 wherein the [2,3]-sigmatropic rearrangement is performed by heating the intermediate of Formula (III) in the solvent.

8. The method of claim 1 wherein the [2-3]-sigmatropic rearrangement is performed by subjecting the intermediate of Formula (III) to microwave irradiation.

9. The method of claim 1 wherein the solvent comprises a C₁-C₃ alcohol.

10. The method of claim 1 wherein the solvent comprises an aqueous buffer.

11. The method of claim 1 wherein the solvent comprises an aqueous buffer mixed with an organic solvent.

12. The method of claim 9 wherein the alcohol comprises methanol.

13. The method of claim 1 wherein the [2,3]-sigmatropic rearrangement is performed in the absence of a phosphine reagent and wherein X is an activating group selected from the group consisting of S-pyridyl, S-heteroaryl, SO₂-aryl, and SO₃Y; Y is an alkali metal ion; Z is S; R¹, R², R³, R⁴, and R⁵ are each independently H or a hydrocarbon moiety; and R is an organic moiety.

14. The method of claim 1 wherein the [2,3]-sigmatropic rearrangement is performed in the presence of an amine.

15. The method of claim 1 wherein the thiol of Formula (II) is a thio-substituted peptide.

16. The method of claim 1 wherein the thiol of Formula (II) is a thio-substituted carbohydrate.

17. (canceled)

18. The method of claim 13 wherein the organic moiety, R, comprises an alkyl group, a substituted-alkyl group, an aryl group, an S-heteroaryl group, a substituted-aryl group, an amino acid, a carbohydrate, a nucleic acid, a peptide nucleic acid, a peptide, or a group consisting of two or more of the foregoing bound together.

19. The method of claim 13 wherein X is S-pyridyl or S-2-benzothiazolyl.

20. (canceled)

21. The method of claim 13 wherein the [2,3]-sigmatropic rearrangement is performed by heating the intermediate of Formula (III) in the solvent, or by subjecting the intermediate of Formula (III) to microwave irradiation.

22-26. (canceled)

27. The method of claim 13 wherein the [2,3]-sigmatropic rearrangement is performed in the presence of an amine.

28-29. (canceled)
